(19) United States
(12) Patent Application Publication
(10) Pub. No.: US 2025/0213583 A1
(43) Pub. Date: Jul. 3, 2025

(54) ANTIVIRAL PHARMACEUTICAL COMPOSITION AND USE THEREOF

(71) Applicant: MITOIMMUNE THERAPEUTICS INC., Gangnam-gu, Seoul (KR)

(72) Inventors: Won-Keun KIM, Chuncheon-si, Gangwon-do (KR); Tae Hun LIM, Guri-si, Gyeonggi-do (KR); Seung-Hwan BAEK, Chuncheon-si, Gangwon-do (KR); Soon-Ha KIM, Gangnam-gu, Seoul (KR)

(21) Appl. No.: 18/850,008

(22) PCT Filed: Mar. 23, 2023
(86) PCT No.: PCT/KR2023/003884

§ 371 Date: Sep. 23, 2024

(30) Foreign Application Priority Data
Mar. 25, 2022 (KR) 10-2022-0037432

(51) Int. Cl. A61K 31/541; A61P 31/14

(52) U.S. Cl. CPC A61K 31/541; A61P 31/14

(57) ABSTRACT

The present invention relates to a pharmaceutical composition for the prevention or treatment of viral infections, comprising a compound of chemical formula 1 or a pharmaceutically acceptable salt thereof as an active ingredient, and a method for the prevention or treatment of viral infections by using same.

### TECHNICAL FIELD

The present invention relates to a pharmaceutical composition for preventing or treating viral infections, including a compound of Chemical Formula 1 or a pharmaceutically acceptable salt thereof, and a method of preventing or treating viral infections using the same.

### BACKGROUND ART

There are numerous viruses on Earth, and new or re-emerging viruses are continuously appearing. Humans can easily be infected with viruses, and viral infections may cause a variety of symptoms from mild symptoms to severe symptoms, and may cause numerous deaths, so viruses pose a major threat not only to the health of humankind but also to the economy, society, and culture. Accordingly, there is an increasing interest in prevalent zoonotic viral infectious diseases such as Middle East respiratory syndrome virus (MERS-CoV), Avian influenza, Ebola virus, and Severe fever with thrombocytopenia syndrome virus (SFTSV), and these viruses have become major issues in our society. In addition, the emergence of unknown viruses existing in nature or new viruses introduced from overseas, such as the Zika virus, may cause unexpected major difficulties for citizens and the health authorities.

Most recently, the outbreak of coronavirus disease-19 (COVID-19), an acute respiratory disease first reported in Wuhan, China in late 2019, has led to the World Health Organization (WHO) declaring it a “pandemic” as confirmed cases continue to be found worldwide.

COVID-19 is known to have originated from bats as a natural host and was caused by a mutation of severe acute respiratory syndrome coronavirus 2 (SARS-CoV-2). Common symptoms include fever, cough, fatigue, dyspnea, olfactory loss, and ageusia, and its rapid spread caused numerous infections and deaths.

Vaccines to prevent viral infections are usually difficult to develop and take a considerable period of time to manufacture and test. In addition, antiviral drugs exhibit their effects by suppressing the replication of viruses in the infected patient's body and relying on the body's immune system, but most antiviral drugs have inconsistent effects, and in many cases, viral infections may clear up before drugs take effect.

Therefore, there is an increasing need for the development of new and effective therapeutics to prevent or treat viral infections.

### RELATED ART DOCUMENTS

### Patent Document

International Patent Publication No. WO 2009/025478

### DISCLOSURE

### Technical Problem

The present invention aims to provide a substance for preventing or treating various types of viral infections.

Accordingly, one object of the present invention is to provide a pharmaceutical composition for preventing or treating viral infections, including a compound of Chemical Formula 1 or a pharmaceutically acceptable salt thereof as an active ingredient.

In addition, another object of the present invention is to provide a method of preventing or treating viral infections using the above ingredient.

### Technical Solution

The present inventors have confirmed that the compound of Chemical Formula 1 or a pharmaceutically acceptable salt thereof exhibits an antiviral effect, thereby completing the present invention.

Accordingly, the present invention provides a pharmaceutical composition for preventing or treating viral infections, including a compound of Chemical Formula 1 or a pharmaceutically acceptable salt thereof as an active ingredient.

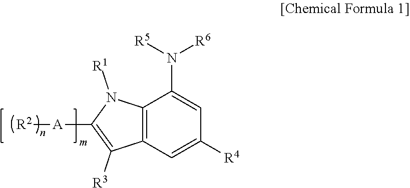

In the above chemical formula,
n is an integer from 1 to 3,
m is 0 or 1,
A represents phenyl,
R¹ is hydrogen or a C₁-C₆ alkyl,
R² represents hydrogen, a halogen, a C₁-C₆ alkoxy, —C₁-C₆ alkylene-OH, —(CH₂)ₚCO₂R⁷, —NHR⁸, —N(H)S(O)₂R⁷, or —NHC(O)R⁷, wherein p is an integer from 0 to 3, R⁷ represents hydrogen or a C₁-C₃ alkyl, and R⁸ represents a C₁-C₃ alkylpiperidinyl or C₁-C₃ alkylsulfonyl,
R³ represents hydrogen, a halogen, a C₁-C₆ alkyl or phenyl or represents-(CH₂)ₚ-heterocycle in which the heterocycle is a 5- to 6-membered ring containing one or two heteroatoms selected from S, N, and O atoms, wherein p is an integer from 0 to 3, provided that when m is 0, R³ is phenyl,
R⁴ is a halogen, a C₁-C₆ alkyl, —C₁-C₆ alkylene-OH, —O-phenyl, —(CH₂)ₚCO₂R⁷, —(CH₂)ₚ-heterocycle in which the heterocycle is a 5- to 6-membered ring containing one or two heteroatoms selected from S, N, and O atoms, or proline-N-carbonyl, wherein p is an integer from 0 to 3, and R⁷ is as defined above,
R⁵ is hydrogen or a C₁-C₆ alkyl, and
R⁶ represents a C₁-C₆ alkyl, a C₃-C₆ cycloalkyl, a heterocycle or —C₁-C₆ alkylene-heterocycle, wherein the heterocycle is a 3- to 8-membered ring containing one to three heteroatoms selected from S, N, and O atoms, R⁶ may be substituted with a C₁-C₆ alkylamine, —C₁-C₆ alkylene-OH, or a C₁-C₆ alkylsulfonyl.

In addition, the present invention provides a method of preventing or treating a viral infection, including administering a compound of Chemical Formula 1 or a pharmaceutically acceptable salt thereof into a subject in need thereof.

### Advantageous Effects

According to the composition or method of the present invention, when cells are treated with the compound of Chemical Formula 1, it can exhibit antiviral efficacy in various cells. In addition, when virus-infected cells are treated with the compound of Chemical Formula 1, it can exhibit antioxidant and anti-inflammatory efficacy, maintain mitochondrial homeostasis, and suppress cell death in the infected cells. Accordingly, the compound of Chemical Formula 1 of the present invention can be effectively used for preventing or treating viral infections.

### DESCRIPTION OF DRAWINGS

FIG. 1A shows a graph illustrating the results of a real-time quantitative polymerase chain reaction (RT-qPCR) analysis for Vero E6 cells according to Experimental Example 1, FIG. 1B shows a graph illustrating the results of a plaque assay for Vero E6 cells according to Experimental Example 1, and FIG. 1C shows an image illustrating Western blot results for Vero E6 cells according to Experimental Example 1.

FIG. 1D shows a graph illustrating the half maximal effective concentration (EC₅₀) value of Example Compound 1 for Vero E6 cells according to Experimental Example 1.

FIGS. 2A and 2B show RT-qPCR graphs by concentration of Example Compound 1 (left) and the EC₅₀ value of Example Compound 1 (right) under the MOI=0.01 condition and the MOI=0.1 condition for hACE2-A549 cells according to Experimental Example 2.

FIG. 2C shows an image illustrating Western blot results for hACE2-A549 cells according to Experimental Example 2, and FIG. 2D shows the plaque assay results.

FIG. 3A shows a graph illustrating the results of next-generation sequencing (NGS) performed according to Experimental Example 3.

FIG. 3B shows a graph illustrating the results of an RT-qPCR analysis for each gene according to Experimental Example 3.

FIG. 4A shows a graph illustrating the results of next-generation sequencing (NGS) performed according to Experimental Example 4.

FIG. 4B shows a graph illustrating the results of an RT-qPCR analysis of heme oxygenase 1 (HMOX1) and Nqo1 according to Experimental Example 4.

FIG. 4C shows a graph illustrating the results of next-generation sequencing (NGS) performed according to Experimental Example 4.

FIGS. 5A and 5B show mitochondrial membrane potential images and intensity graphs using JC-1 staining according to Experimental Example 5, FIG. 5C shows membrane potential images using MitoTracker staining according to Experimental Example 5, and FIG. 5D shows Western blot images of mitochondrial dynamic factors according to Experimental Example 5.

FIGS. 6A and 6B show Western blot images of mitochondrial cell death factors at 24 hours post infection (hpi) and 48 hpi according to Experimental Example 6.

FIGS. 7A, 7B, 7C, 7D, and 7E show graphs illustrating the results of RT-qPCR analyses by virus type according to Experimental Example 7.

### MODES OF THE INVENTION

Hereinafter, the present invention will be described in detail.

Meanwhile, each description and embodiment disclosed in the present invention may be applied to other descriptions and embodiments. That is, all combinations of the various elements disclosed in the present invention are interpreted as falling within the scope of the present invention. In addition, the scope of the present invention is not limited by the specific description described below.

When one part “includes” a certain component, unless particularly stated otherwise, other components may be further included, rather than excluded.

The present invention provides a composition for preventing or treating viral infections, which includes a compound of Chemical Formula 1, or a pharmaceutically acceptable salt thereof as an active ingredient.

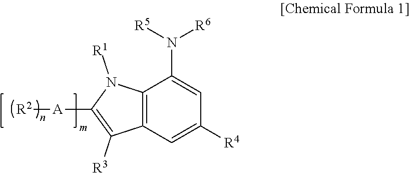

In this formula,
n may be an integer from 1 to 3,
m may be 0 or 1,
A may be phenyl,
R¹ may be hydrogen or C₁-C₆ alkyl,
R² may be hydrogen, halogen, C₁-C₆ alkoxy, —C₁-C₆ alkylene-OH, —(CH₂)ₚCO₂R⁷, —NHR⁸, —N(H)S(O)₂R⁷, or —NHC(O)R⁷, wherein p is an integer from 0 to 3, R⁷ is hydrogen or C₁-C₃ alkyl, and R⁸ is C₁-C₃ alkylpiperidinyl or C₁-C₃ alkylsulfonyl,
R³ may be hydrogen, halogen, C₁-C₆ alkyl, phenyl, or —(CH₂)ₚ-heterocycle, in which the heterocycle is a 5- or 6-membered ring including 1 or 2 hetero atoms selected from S, N, and O atoms, wherein p is an integer from 0 to 3, provided that when m is 0, R³ is phenyl,
R⁴ may be halogen, C₁-C₆ alkyl, —C₁-C₆ alkylene-OH, —O-phenyl, —(CH₂)ₚCO₂R⁷,
   (CH₂)ₚ-heterocycle, in which the heterocycle is a 5- or 6-membered ring including 1 or 2 hetero atoms selected from S, N, and O atoms, or proline-N-carbonyl wherein p is an integer from 0 to 3, and R⁷ is the same as defined above,
R⁵ may be hydrogen or C₁-C₆ alkyl,
R⁶ may be C₁-C₆ alkyl, C₃-C₆ cycloalkyl, heterocycle, or —C₁-C₆ alkylene-heterocycle, in which the heterocycle is a 3- to 8-membered ring including 1 to 3 hetero atoms selected from S, N, and O atoms, and R⁶ may be substituted with C₁-C₆ alkylamine, —C₁-C₆ alkylene-OH, or C₁-C₆ alkylsulfonyl.

The compound of Chemical Formula 1 according to the present invention can exhibit antiviral efficacy against various types of viruses, and can exhibit an effect of suppressing the amount of viruses or the amount of viral replication. In addition, the present invention confirmed that it can exhibit antioxidant and anti-inflammatory effects, maintain mitochondrial homeostasis, and suppress cell death for cells that have become abnormal due to viral infection. Thus, the present invention identifies a novel use of the compound of Chemical Formula 1.

The compound of Chemical Formula 1 of the present invention may be used in the form of a pharmaceutically acceptable salt thereof. Particularly, the pharmaceutically acceptable salt may be an acid addition salt formed by a free acid. Here, the acid addition salt may be obtained from inorganic acids such as hydrochloric acid, nitric acid, phosphoric acid, sulfuric acid, hydrobromic acid, hydroiodic acid, nitrous acid or phosphorous acid, non-toxic organic acids such as aliphatic mono- and di-carboxylates, phenyl-substituted alkanoates, hydroxy alkanoates and alkane dioates, aromatic acids, aliphatic, and aromatic sulfonic acids, and organic acids such as trifluoroacetic acid, acetate, benzoic acid, citric acid, lactic acid, maleic acid, gluconic acid, methanesulfonic acid, 4-toluenesulfonic acid, tartaric acid, and fumaric acid. Types of such pharmaceutically acceptable salts may include sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, and propionate.

The composition of the present invention may include not only the compound of Chemical Formula 1 and a pharmaceutically acceptable salt thereof, but also any salt, isomer, hydrate, and/or solvate, which can be prepared by a conventional method.

The “isomer” used herein may refer to compounds of the present invention, which have the same chemical formula or molecular formula, but are structurally or sterically different, or salts thereof. These isomers include structural isomers such as tautomers, R or S isomers with asymmetric carbon centers, geometric isomers (trans, cis), and optical isomers (enantiomers). All these isomers and mixtures thereof are also included within the scope of the present invention.

The “hydrate” used herein may mean a compound of the present invention, which includes a stoichiometric or non-stoichiometric amount of water, which is bound by a non-covalent intermolecular force, or a salt thereof. A hydrate of the compound represented by Chemical Formula 1 of the present invention may include a stoichiometric or non-stoichiometric amount of water, which is bound by a non-covalent intermolecular force. The hydrate may contain 1 eq or more, and preferably, 1 to 5 eq of water. Such a hydrate may be prepared by crystallizing a compound represented by Chemical Formula 1 of the present invention, an isomer thereof, or a pharmaceutically acceptable salt thereof from water or a water-containing solvent.

The “solvate” used herein may refer to a compound of the present invention, which includes a stoichiometric or non-stoichiometric amount of solvent, which is bound by a non-covalent intermolecular force, or a salt thereof. Preferred solvents therefor include solvents that are volatile, non-toxic, and/or suitable for administration to humans.

The term “alkyl” used herein refers to an aliphatic hydrocarbon radical. Alkyl may be “saturated alkyl” that does not include an alkenyl or alkynyl moiety, or “unsaturated alkyl” that includes at least one alkenyl or alkynyl moiety, and have, unless defined otherwise, 1 to 20 carbon atoms. Alkyl, alkenyl, and alkynyl may mean linear or branched acyclic hydrocarbons.

The term “alkylene” may mean a bivalent hydrocarbon group in which a radical is additionally formed from the alkyl, and include, for example, methylene, ethylene, propylene, butylene, and isobutylene, but the present invention is not limited thereto.

The term “alkoxy” refers to, unless defined otherwise, alkyl-oxy having 1 to 10 carbon atoms.

The term “cycloalkyl” refers to, unless defined otherwise, a saturated aliphatic 3- to 10-membered ring. Typical cycloalkyl groups include cyclopropyl, cyclobutyl, cyclopentyl, and cyclohexyl, but the present invention is not limited thereto.

The term “heterocycle” refers to, unless defined otherwise, a 3- to 10-membered ring, preferably, a 4- to 8-membered ring, and more preferably, a 5- to 6-membered ring, which includes 1 to 3 hetero atoms selected from the group consisting of N, O, and S, may be fused with benzo or C₃-C₈ cycloalkyl, is saturated, or has 1 or 2 double bonds. In addition, the “heterocycle” may be used interchangeably with the term “heterocyclyl.” Examples of heterocycles may include pyrroline, pyrrolidine, imidazoline, imidazolidine, pyrazoline, pyrazolidine, pyran, piperidine, morpholine, thiomorpholine, piperazine, and hydrofuran, but the present invention is not limited thereto.

Other terms and abbreviations used herein, unless defined otherwise, can be interpreted as generally understood by those of ordinary skill in the art to which the present invention pertains.

In one embodiment of the present invention, in the compound of Chemical Formula 1,
R³ may be hydrogen, halogen, phenyl, or —(CH₂)ₚ-heterocycle in which the heterocycle is morpholino or piperazinonyl. Here, p is an integer of 0 or 1, provided that when m is 0, R³ may be phenyl.

In one embodiment of the present invention, in the compound of Chemical Formula 1,
R⁴ may be halogen, C₁-C₃ alkyl, —C₁-C₃ alkylene-OH, —O-phenyl, —(CH₂)ₚCO₂-ethyl,
   (CH₂)ₚ-heterocycle in which the heterocycle is thiomorpholino, morpholino, piperazinonyl, or pyrrolidinyl, or proline-N-carbonyl, wherein p may be an integer of 0 or 1.

In one embodiment of the present invention, in the compound of Chemical Formula 1,
R⁵ may be hydrogen or C₁-C₃ alkyl,
R⁶ may be C₁-C₃ alkyl, C₃-C₆ cycloalkyl, heterocycle, or —C₁-C₃ alkylene-heterocycle, wherein the heterocycle is tetrahydro-2H-pyran, or piperidinyl. When R⁶ is heterocycle or —C₁-C₃ alkylene-heterocycle, the heterocycle may be substituted with C₁-C₆ alkylamine, —C₁-C₆ alkylene-OH, or C₁-C₆ alkylsulfonyl.

In the present invention, examples of the compound of Chemical Formula 1 may include compounds 1 to 32 listed in Table 1 below, or pharmaceutically acceptable salts thereof.

TABLE 1

| No. | Structure | Name of Compound |
|---|---|---|
| 1 | 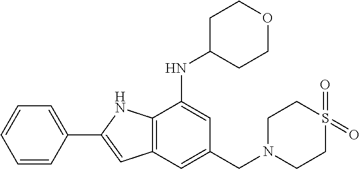 [SMILES: O=S1(=O)CCN(Cc2cc(NC3CCOCC3)c3[nH]c(-c4ccccc4)cc3c2)CC1] | 5-[(1,1-dioxido-4-thiomorpholinyl) methyl]-2-phenyl-N- (tetrahydro-2H-pyran-4-yl)- 1H-indol-7-amine |
| 2 | 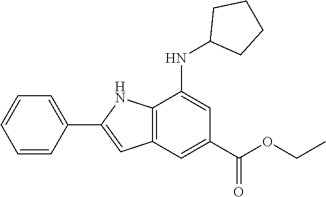 [SMILES: CCOC(=O)c1cc(NC2CCCC2)c2[nH]c(-c3ccccc3)cc2c1] | ethyl 7-(cyclopentylamino)- 2-phenyl-1H-indol-5- carboxylate |
| 3 | 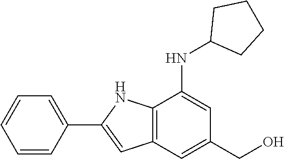 [SMILES: OCc1cc(NC2CCCC2)c2[nH]c(-c3ccccc3)cc2c1] | (7-(cyclopentylamino)-2- phenyl-1H-indol-5- yl)methanol |
| 4 | 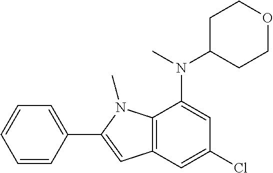 [SMILES: CN(c1cc(Cl)cc2cc(-c3ccccc3)n(C)c12)C1CCOCC1] | 5-chloro-N,1-dimethyl-2- phenyl-N-(tetrahydro-2H- pyran-4-yl)-1H-indol-7-amine |
| 5 | 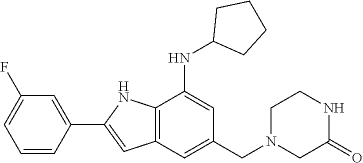 [SMILES: O=C1CN(Cc2cc(NC3CCCC3)c3[nH]c(-c4cccc(F)c4)cc3c2)CCN1] | 4-((7-(cyclopentylamino)-2- (3-fluorophenyl)-1H-indol- 5-yl)methyl)piperazin-2-one |
| 6 | 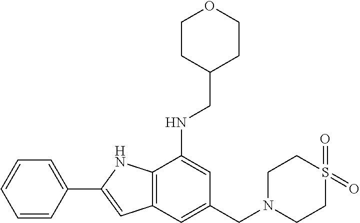 [SMILES: O=S1(=O)CCN(Cc2cc(NCC3CCOCC3)c3[nH]c(-c4ccccc4)cc3c2)CC1] | 4-((2-phenyl-7-(((tetrahydro- 2H-pyran-4- yl)methyl)amino)-1H-indol-5- yl)methyl)thiomorpholine 1,1-dioxide |
| 7 | 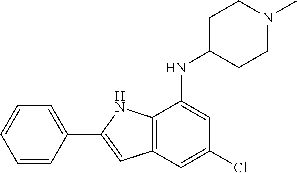 [SMILES: CN1CCC(Nc2cc(Cl)cc3cc(-c4ccccc4)[nH]c23)CC1] | 5-chloro-N-(1-methylpiperidin- 4-yl)-2-phenyl-1H- indol-7-amine |
| 8 | 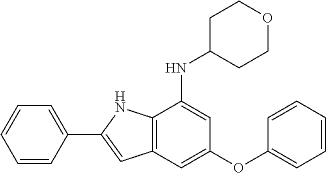 [SMILES: c1ccc(Oc2cc(NC3CCOCC3)c3[nH]c(-c4ccccc4)cc3c2)cc1] | 5-phenoxy-2-phenyl-N-(tetrahydro- 2H-pyran-4-yl)-1H- indol-7-amine |
| 9 | 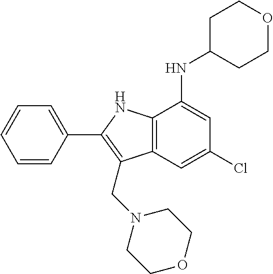 [SMILES: Clc1cc(NC2CCOCC2)c2[nH]c(-c3ccccc3)c(CN3CCOCC3)c2c1] | 5-chloro-3-(morpholinomethyl)- 2-phenyl-N- (tetrahydro-2H-pyran-4-yl)- 1H-indol-7-amine |
| 10 | 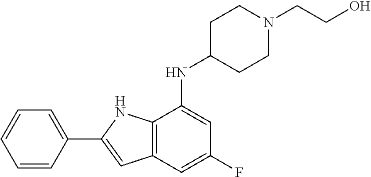 [SMILES: OCCN1CCC(Nc2cc(F)cc3cc(-c4ccccc4)[nH]c23)CC1] | 2-(4-((5-fluoro-2-phenyl-1H-indol-7- yl)amino)piperidin-1-yl)ethan-1-ol |
| 11 | 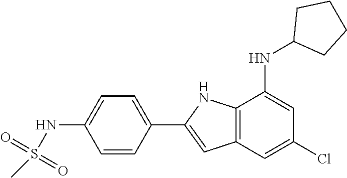 [SMILES: CS(=O)(=O)Nc1ccc(-c2cc3cc(Cl)cc(NC4CCCC4)c3[nH]2)cc1] | N-(4-(5-chloro-7- (cyclopentylamino)-1H-indol-2- yl)phenyl)methanesulfonamide |
| 12 | 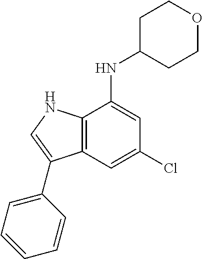 [SMILES: Clc1cc(NC2CCOCC2)c2[nH]cc(-c3ccccc3)c2c1] | 5-chloro-3-phenyl-N-(tetrahydro- 2H-pyran-4-yl)-1H- indol-7-amine |
| 13 | 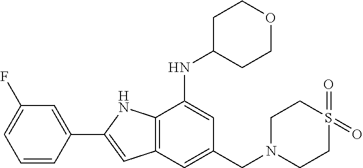 [SMILES: O=S1(=O)CCN(Cc2cc(NC3CCOCC3)c3[nH]c(-c4cccc(F)c4)cc3c2)CC1] | 4-((2-(3-fluorophenyl)-7- ((tetrahydro-2H-pyran-4- yl)amino)-1H-indol-5-yl) methyl)thiomorpholine 1,1- dioxide |
| 14 | 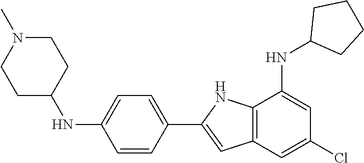 [SMILES: CN1CCC(Nc2ccc(-c3cc4cc(Cl)cc(NC5CCCC5)c4[nH]3)cc2)CC1] | 5-chloro-N-cyclopentyl-2-(4- ((1-methylpiperidin-4- yl)amino)phenyl)-1H-indol- 7-amine |
| 15 | 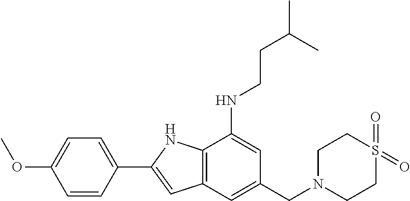 [SMILES: COc1ccc(-c2cc3cc(CN4CCS(=O)(=O)CC4)cc(NCCC(C)C)c3[nH]2)cc1] | 4-((7-(isopentylamino)-2- (4-methoxyphenyl)-1H-indol- 5-yl)methyl)thiomorpholine 1,1-dioxide |
| 16 | 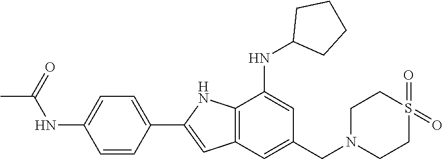 [SMILES: CC(=O)Nc1ccc(-c2cc3cc(CN4CCS(=O)(=O)CC4)cc(NC4CCCC4)c3[nH]2)cc1] | N-(4-(7- (cyclopentylamino)-5-((1,1- dioxidomorpholino) methyl)-1H-indol-2- yl)phenyl)acetamide |
| 17 | 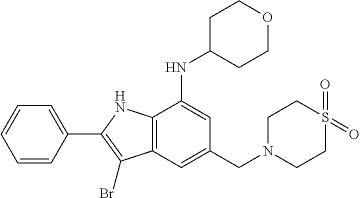 [SMILES: O=S1(=O)CCN(Cc2cc(NC3CCOCC3)c3[nH]c(-c4ccccc4)c(Br)c3c2)CC1] | 4-((3-bromo-2-phenyl-7- ((tetrahydro-2H-pyran-4- yl)amino)-1H-indol-5-yl) methyl)thiomorpholine 1,1- dioxide |
| 18 | 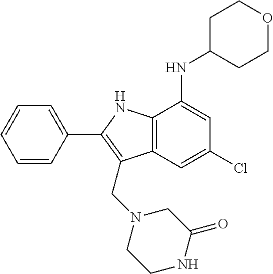 [SMILES: O=C1CN(Cc2c(-c3ccccc3)[nH]c3c(NC4CCOCC4)cc(Cl)cc23)CCN1] | 4-((5-chloro-2-phenyl-7- ((tetrahydro-2H-pyran-4- yl)amino)-1H-indol-3- yl)methyl)piperazin-2-one |
| 19 | 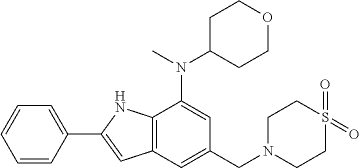 [SMILES: CN(c1cc(CN2CCS(=O)(=O)CC2)cc2cc(-c3ccccc3)[nH]c12)C1CCOCC1] | 4-((7-(methyl(tetrahydro- 2H-pyran-4-yl)amino)-2- phenyl-1H-indol-5-yl) methyl)thiomorpholine 1,1- dioxide |
| 20 | 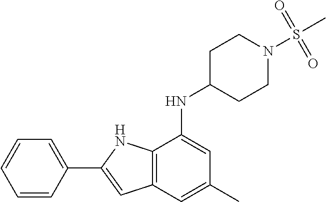 [SMILES: Cc1cc(NC2CCN(S(C)(=O)=O)CC2)c2[nH]c(-c3ccccc3)cc2c1] | 5-methyl-N-(1- (methylsulfonyl)piperidin-4-yl)-2- phenyl-1H-indol-7-amine |
| 21 | 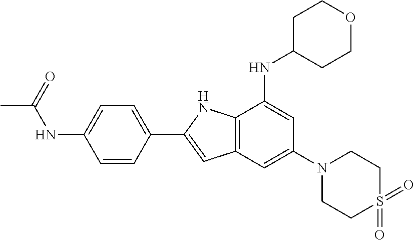 [SMILES: CC(=O)Nc1ccc(-c2cc3cc(N4CCS(=O)(=O)CC4)cc(NC4CCOCC4)c3[nH]2)cc1] | N-(4-(5-(1,1- dioxidothiomorpholino)-7-((tetrahydro- 2H-pyran-4-yl)amino)- 1H-indol-2-yl)phenyl)acetamide |
| 22 | 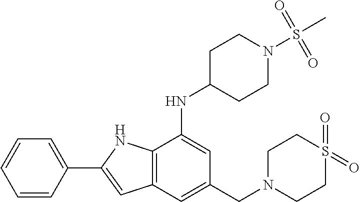 [SMILES: CS(=O)(=O)N1CCC(Nc2cc(CN3CCS(=O)(=O)CC3)cc3cc(-c4ccccc4)[nH]c23)CC1] | 4-((7-((1-methylsulfonyl) piperidin-4-yl)amino)-2- phenyl-1H-indol-5-yl) methyl)thiomorpholine 1,1- dioxide |
| 23 | 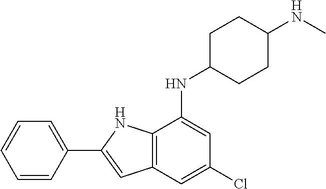 [SMILES: CNC1CCC(Nc2cc(Cl)cc3cc(-c4ccccc4)[nH]c23)CC1] | N¹-(5-chloro-2-phenyl- 1H-indol-7-yl)-N⁴- methylcyclohexane-1,4-diamine |
| 24 | 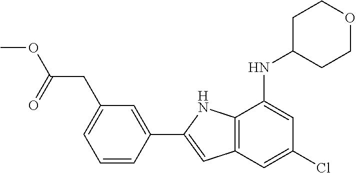 [SMILES: COC(=O)Cc1cccc(-c2cc3cc(Cl)cc(NC4CCOCC4)c3[nH]2)c1] | methyl 2-(3-(5-chloro-7- ((tetrahydro-2H-pyran-4- yl)amino)-1H-indol- 2-yl)phenyl)acetate |
| 25 | 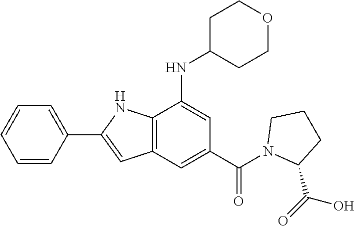 [SMILES: O=C(O)[C@H]1CCCN1C(=O)c1cc(NC2CCOCC2)c2[nH]c(-c3ccccc3)cc2c1] | (2-phenyl-7-((tetrahydro- 2H-pyran-4-yl)amino)-1H- indol-5-carbonyl)-D-proline |
| 26 | 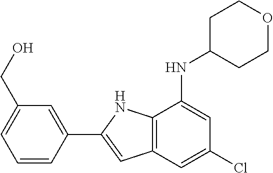 [SMILES: OCc1cccc(-c2cc3cc(Cl)cc(NC4CCOCC4)c3[nH]2)c1] | (3-(5-chloro-7-((tetrahydro-2H- pyran-4-yl)amino)-1H- indol-2-yl)phenyl)methanol |
| 27 | 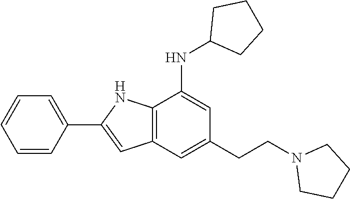 [SMILES: c1ccc(-c2cc3cc(CCN4CCCC4)cc(NC4CCCC4)c3[nH]2)cc1] | N-cyclopentyl-2-phenyl-5- (2-(pyrrolidin-1-yl)ethyl)- 1H-indol-7-amine |
| 28 | 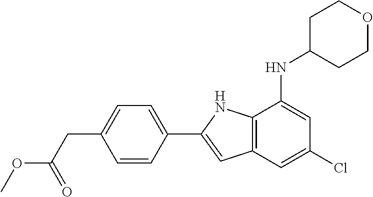 [SMILES: COC(=O)Cc1ccc(-c2cc3cc(Cl)cc(NC4CCOCC4)c3[nH]2)cc1] | methyl 2-(4-(5-chloro-7- ((tetrahydro-2H-pyran-4- yl)amino)-1H-indol-2-yl) phenyl)acetate |
| 29 | 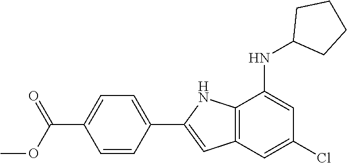 [SMILES: COC(=O)c1ccc(-c2cc3cc(Cl)cc(NC4CCCC4)c3[nH]2)cc1] | methyl 4-(5-chloro-7- (cyclopentylamino)-1H-indol-2- yl)benzoate |
| 30 | 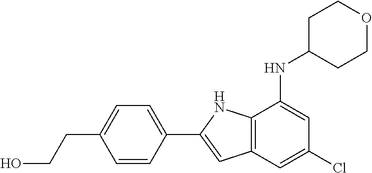 [SMILES: OCCc1ccc(-c2cc3cc(Cl)cc(NC4CCOCC4)c3[nH]2)cc1] | 2-(4-(5-chloro-7- ((tetrahydro-2H-pyran-4-yl)amino)- 1H-indol-2-yl)phenyl)ethan-1-ol |
| 31 | 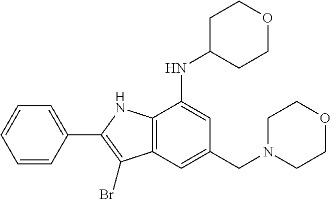 [SMILES: Brc1c(-c2ccccc2)[nH]c2c(NC3CCOCC3)cc(CN3CCOCC3)cc12] | 3-bromo-5- (morpholinomethyl)-2-phenyl-N- (tetrahydro-2H-pyran-4-yl)- 1H-indol-7-amine |
| 32 | 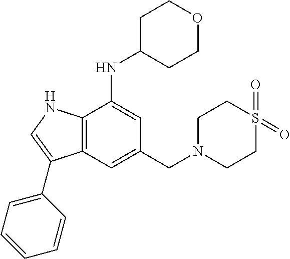 [SMILES: O=S1(=O)CCN(Cc2cc(NC3CCOCC3)c3[nH]cc(-c4ccccc4)c3c2)CC1] | 4-((3-phenyl-7-((tetrahydro- 2H-pyran-4-yl)amino)-1H- indol-5-yl)methyl) thiomorpholine 1,1-dioxide |

In one embodiment of the present invention, the compound of Chemical Formula 1 may be a compound of Chemical Formula 2.

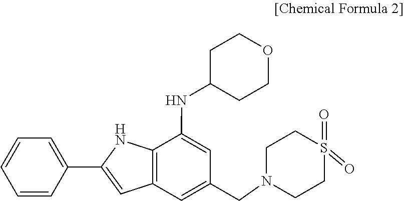 [SMILES: O=S1(=O)CCN(Cc2cc(NC3CCOCC3)c3[nH]c(-c4ccccc4)cc3c2)CC1]

The compound of Chemical Formula 1 of the present invention or a pharmaceutically acceptable salt thereof has an antiviral effect and can prevent or treat diseases caused by viral infections. Here, the type of virus is not particularly limited.

In one specific embodiment of the present invention, the pharmaceutical composition may be for the prevention or treatment of one or more viral infections selected from the group consisting of severe acute respiratory syndrome coronavirus type 2 (SARS-CoV-2), severe acute respiratory syndrome coronavirus type 1 (SARS-CoV-1), Zika virus (ZIKV), Gamak virus (GAKV), respiratory syncytial virus (RSV), vaccinia virus (VACV), influenza virus, flavivirus, adenovirus (AdV), Middle East respiratory syndrome coronavirus (MERS-CoV), herpes virus, Japanese encephalitis virus (JEV), Epstein-Barr virus (EBV), Ebola virus (EBOV), rhinovirus, chikungunya virus (CHIKV), hepatitis A virus (HAV), hepatitis B virus (HBV), hepatitis C virus (HCV), rotavirus, astrovirus, hantavirus including Seoul virus (SEOV), dengue virus, severe fever with thrombocytopenia syndrome virus (SFTSV), human immunodeficiency virus (HIV), West Nile virus (WNV), and yellow fever virus, and variants thereof.

In one specific embodiment of the present invention, the pharmaceutical composition may be for the prevention or treatment of one or more viral infections selected from the group consisting of SARS-CoV-2, SARS-CoV-2 B.1 (Wuhan), SARS-CoV-2 B.1.617.2 (delta), SARS-CoV-2 BA.1 (omicron), Seoul virus (SEOV), Zika virus (ZIKV), and VACV.

In one specific embodiment of the present invention, the pharmaceutical composition may be for the prevention or treatment of a disease caused by SARS-CoV-2 infections, specifically, coronavirus disease 2019 (COVID-19), and more specifically, may be for the prevention or treatment of SARS-CoV-2 B.1 (Wuhan), SARS-CoV-2 B.1.617.2 (delta), and SARS-CoV-2 BA. 1 (omicron) virus infections.

In the present invention, “SARS-CoV-2” encompasses all SARS-CoV-2 variants, and may include, for example, all variants referred to as variants B.1, B.1.617.2, and BA.1. These variants may be due to mutations (e.g., additions, substitutions, and/or deletions of amino acids) in the spike protein.

In the present invention, “antiviral” or “virus inhibition” means the ability to inhibit a virus particle from infecting a host cell, or to inhibit a virus particle from replicating or proliferating in a host cell.

In the present specification, “infection” means a state in which a pathogenic microorganism has invaded the body of a host organism and proliferated in the body.

In the present specification, “viral infection” means a disease caused by a viral infection, and symptoms or signs thereof are not limited as long as the cause of the disease is a viral infection

In one specific embodiment of the present invention, the compound of Chemical Formula 1 or a pharmaceutically acceptable salt thereof may have an EC₅₀ of 0.01 to 10 μM against a virus.

The compound of Chemical Formula 1 of the present invention or a pharmaceutically acceptable salt thereof may inhibit viral replication in virus-infected cells. In addition, it may inhibit the occurrence of inflammation, the occurrence of oxidative stress, and cell death in virus-infected cells.

In one specific embodiment of the present invention, the pharmaceutical composition may inhibit the expression of one or more genes selected from the group consisting of Ifnb, Tnfα, Il-6, Ifit1, and Ifit2, which is increased due to viral infection, and may increase the expression of one or more genes selected from the group consisting of HMOX1 and Nqo1, which is inhibited due to viral infection.

In one specific embodiment of the present invention, the pharmaceutical composition may inhibit the expression of one or more genes selected from the group consisting of MLKL, p-MLKL, caspase-3, cleaved caspase-3, MFN1, and MFN2, which are mitochondrial homeostasis-related genes.

The “treatment” used herein refers to stopping or delaying the progression of a disease when used in subjects exhibiting symptoms of a disease, and the “prevention” used herein refers to stopping or delaying the onset of disease when used in subjects that do not exhibit symptoms but are at high risk for such disease.

The “pharmaceutical composition” used herein may include a pharmaceutically acceptable carrier as needed, along with the compound of the present invention.

The compound of Chemical Formula 1 according to the present invention may be administered in a variety of oral and parenteral dosage forms in clinical administration, and may be formulated with diluents or excipients, such as a filler, an extender, a binder, a wetting agent, a disintegrant, and a surfactant.

Solid preparations for oral administration include tablets, pills, powders, granules, capsules, and troches, and such solid preparations are prepared by mixing at least one excipient, such as starch, calcium carbonate, sucrose, lactose, or gelatin with one or more of the compounds of the present invention. In addition, in addition to simple excipients, lubricants such as magnesium stearate, talc, etc. are also used. Liquid preparations for oral administration include suspensions, liquids for internal use, emulsions, and syrups, and may further include various types of excipients, for example, a wetting agent, a sweetener, a fragrance and a preservative, other than a commonly-used simple diluent such as water, or liquid paraffin.

Formulations for parenteral administration may include sterilized aqueous solutions, non-aqueous solvents, suspensions, emulsions, freeze-dried formulations, or suppositories. As the non-aqueous solvent or suspension, propylene glycol, polyethylene glycol, a vegetable oil such as olive oil, or an injectable ester such as ethyl oleate may be used. As a suppository base, Witepsol, Tween 61, cacao butter, laurin butter, glycerol, or gelatin may be used.

In addition, an effective dose of the compound of Chemical Formula 1 of the present invention for the human body may vary depending on a patient's age, body weight, gender, administration type, health condition, and severity of a disease, and is generally approximately 0.001 to 100 mg/kg/day, and preferably 0.01 to 35 mg/kg/day. Based on an adult patient weighing 70 kg, the composition of Chemical Formula 1 may be administered at a dose of generally 0.07 to 7000 mg/day, and preferably, 0.7 to 2500 mg/day, and may be administered once or in divided portions several times a day at regular intervals depending on a doctor's or pharmacist's judgement.

The term “subject” used herein include vertebrates such as mammals including humans and livestock, and birds, in which an inflammatory disease can be alleviated, prevented, or treated by administration of a pharmaceutical composition of the present invention, but the present invention is not limited thereto.

The “administration” used herein refers to introducing a given material into a human or animal by any proper method, and the administration route of a composition for prevention or treatment according to the present invention may be oral or parenteral administration via any general route as long as the composition can reach desired tissue.

The pharmaceutical composition of the present invention can be administered not only to patients showing symptoms or signs of infection, but also to patients who are likely to be infected.

The pharmaceutical composition of the present invention can be used in combination with an antiviral agent that is already commonly used.

The numerical values described above in the specification should be interpreted as including the equivalent range unless specified otherwise.

Hereinafter, the present invention will be described in detail through the following experimental examples. However, the following experimental examples are only intended to illustrate the present invention, and the content of the present invention is not limited by the following experimental examples. In addition, since these experimental examples are only intended to help understand the present invention, the scope of the present invention is not limited by them in any way.

### EXAMPLES

### Experimental Method

### (1) Preparation of Example Compound 1 and Cell Culture

In the present example, as a representative example of the compound of Chemical Formula 1, 5-[(1,1-dioxido-4-thiomorphorinyl)methyl]-2-phenyl-N-(tetrahydro-2H-pyran-4-yl)-1H-indol-7-amine (hereinafter referred to as ‘Example Compound 1’ or ‘Compound 1’) was used.

Vero E6 cells and Calu-3 cells were cultured in Dulbecco's modified Eagle's medium (DMEM; Gibco, cat #2003610) including 1% 10 mM hydroxyethyl piperazine ethane sulfonic acid (HEPES) in 0.85% NaCl, 1% Antibiotic-Antimycotic (Gibco™, cat #15240062; 10 U/mL penicillin, 100 μg/mL streptomycin, 0.25 μg/mL Fungizone™), and 10% heat-inactivated fetal bovine serum (FBS; Gibco, cat #10082147). The cells were cultured under 37° C. and 5% CO₂ conditions, and subcultured to eliminate overpopulation caused by cell proliferation.

A549 cells expressing the human ACE2 receptor (hereinafter referred to as ‘hACE2-A549 cells’) were cultured in DMEM medium supplemented with 10% FBS, 1% 10 mM HEPES in 0.85% NaCl, 100 U/mL penicillin, 100 μg/mL The cells were cultured under 37° C. streptomycin, and 100 μg/mL Normocin™ and 5% CO₂ conditions and subcultured in a growth medium supplemented with 0.5 μg/mL of puromycin every two passages.

### (2) In Vitro Viral Infection

Vero E6 cells, Calu-3 cells (1×10⁶ cells per well), and hACE2-A549 cells (0.5×10⁶ cells per well) were seeded in 6-well plates and placed in an incubator at 37° C. and 5% CO₂ conditions overnight. When the cells reached approximately 80% density, they were washed twice with pre-heated phosphate-buffered saline (PBS; Gibco™ cat #70011069), 1 mL of 2% FBS medium was added, and the cells were infected at the following multiplicity of infection [MOI] values at 37° C. for two hours (MOI=0.01 for Vero E6 cells, MOI=0.1 for hACE2-A549 cells, and MOI=1 for Calu-3 cells). The infected plate was shaken every 15 minutes to efficiently distribute the infectious material. After two hours of virus adsorption, Example Compound 1 was administered. Next, the supernatants and cells were harvested at 24 hours post infection (hpi).

### (3) Plaque Assay

Vero E6 cells were seeded in 6-well plates and incubated overnight under 37° C. and 5% CO₂ conditions. The cells were washed with preheated phosphate buffered saline (PBS) and infected with a 10-fold dilution of the virus supernatant using serum-free medium. For 90 minutes after infection, the plate was shaken every 15 minutes to adsorb the virus, and the cells were covered with 3 mL of overlay medium (DMEM/F12 medium) containing 0.6% purified agar. Thereafter, the cells were cultured at 37° C. and 5% CO₂ for four days and fixed with 3.7% formaldehyde for 24 hours. After removing the overlay agar medium, the plate was stained with 0.1% crystal violet containing 20% methanol for 30 minutes. Here, conditions such as incubation time after infection may be varied depending on the type of virus. Specifically, in the case of the SARS-CoV-2 plaque assay, the incubation was performed for four days in a medium mixed with semi-solid agar under 37° C. and 5% CO₂ conditions, and Zika Virus (ZIKV) was incubated for five days, and vaccinia virus (VACV) was incubated for three days without the addition of semi-solid agar. Thereafter, the virus quantity was measured by counting the number of plaques formed.

### (4) RT-qPCR

The antiviral efficacy was confirmed at the genetic level by observing the amount of viral replication through the measurement of viral RNA genes through RT-qPCR.

RNA extraction was performed using Trizol (Ambion, cat #15596026). First, cells were dissolved in 1 mL of Trizol and then transferred to a 1.5 mL tube. Thereafter, 200 μL of chloroform (EMSURE, cat #1.02445.1000) was added, mixed evenly, and centrifuged at 4° C. and 13,000 rpm for 15 minutes. After centrifugation, the supernatant was transferred to a new tube, and 600 μL of isopropanol (EMSURE, cat #1.09634.1011) and 5 μL of linear acrylamide (Ambion, cat #AM9520) were added and mixed. After centrifugation at 4° C. and 13,000 rpm for 10 minutes, the supernatant was removed, 1 mL of 75% ethanol (EMSURE, cat #1.00983.1011) was added, and centrifugation was performed again at 4° C. and 13,000 rpm for 10 minutes. After removing all the supernatant, the resulting product was dried in air and the RNA pellet was dissolved and diluted in 20 μL of diethylpyrocarbonate (DEPC)-treated water (Ambion, cat #AM9906). Thereafter, the RNA concentration was measured using a spectrophotometer Nanodrop 2000 (Thermo Scientific).

For complementary DNA (cDNA) synthesis, a high-capacity RNA-to-cDNA kit (Applied Biosystems, cat #4387949) was used. 5 μL of a 2× buffer, 0.5 μL of reverse transcriptase (RT) enzyme, DEPC-treated water, and template RNA were added to a volume of 4.5 μL (total of 10 μL), and a thermal cycler (Thermo Scientific) was used to synthesize cDNA through the PCR cycle of Step 1 (37.0° C., 60 minutes, 1 cycle) and Step 2 (95.0° C., 5 minutes, 1 cycle).

RT-qPCR was performed using power SYBR Green PCR Master Mix (Applied Biosystems, cat #4367659). The prepared cDNA template was diluted 1:40 with DEPC-treated water. 5 μL of SYBR green PCR Master Mix, 1 μL of primers for qPCR, and 4 μL of cDNA template were added and mixed, and then RT-qPCR was performed using a Quantstudio3 (Thermo Scientific) machine. Here, the primer sequences used are shown in Tables 2 to 4 below. Table 2 shows the primer sequences required for the detection of SARS-CoV-2, Table 3 shows the primer sequences required for the detection of various viruses including SARS-CoV-2, Table 4 shows the primer sequences of human genes, and Table 5 shows the conditions for performing RT-qPCR.

In the drawings showing the RT-qPCR results, “+” means that the material was treated, and “−” means that the material was not treated

TABLE 2

| | | | SEQ |
|---|---|---|---|
| | | Direc- | ID |
| Target | Sequence | tion | NO. |
| N gene | 5′-CACATTGGCACCCGCAATC-3′ | Forward | 1 |
| | 5′-GAGGAACGAGAAGAGGCTTG-3′ | Reverse | 2 |
| E gene | 5′-ACAGGTACGTTAATAGTTAATAGCGT- | Forward | 3 |
| | 3′ | | |
| | 5′-ATATTGCAGCAGTACGCACACA-3′ | Reverse | 4 |
| RdRp | 5′-GTGARATGGTCATGTGTGGCGG-3′ | Forward | 5 |
| gene | 5′-CARATGTTAAASACACTATTAGCATA- | Reverse | 6 |
| | 3′ | | |

TABLE 3

| | | | | SEQ |
|---|---|---|---|---|
| | Target | | Direc- | ID |
| Virus | gene | Sequence | tion | NO. |
| SARS- | N | 5′-CAC ATT GGC ACC CGC | Forward | 7 |
| CoV-2 | | AAT C-3′ | | |
| | | 5′-GAG GAA CGA GAA GAG | Reverse | 8 |
| | | GCT TG-3′ | | |
| ZIKV | NS1 | 5′-CRA CTA CTG CAA GYG | Forward | 9 |
| | | GAA GG-3′ | | |
| | | 5′-GCC TTA TCT CCA TTC | Reverse | 10 |
| | | CAT ACC-3′ | | |
| SEOV | NP | 5′-TGG CAC TAG CAA AAG | Forward | 11 |
| | | ACT GG-3′ | | |
| | | 5′-CAG ATA AAC TCC CAG | Reverse | 12 |
| | | CAA TAG GA-3′ | | |
| VACV | E9 | 5′-CGC CTA AGA GTT GCA | Forward | 13 |
| | | CAT CCA-3′ | | |
| | | 5′-CTC TGC TCC ATT TAG | Reverse | 14 |
| | | TAC CGA TTC T-3′ | | |

Here, N stands for Nucleocapsid protein, NS1 stands for Non-structure Protein 1, NP stands for Nucleoprotein, and E9 stands for DNA polymerase.

TABLE 4
Human related gene

| | | | SEQ |
|---|---|---|---|
| | | Direc- | ID |
| Gene | Sequence | tion | NO. |
| HMOX1 | 5′-CGG ATG GAG CGT CCG CAA | Forward | 15 |
| | CC-3′ | | |
| | 5′-TCA CCA GCT TGA AGC CGT | Reverse | 16 |
| | CTC G-3′ | | |
| Nqo1 | 5′-CCC CGG ACT GCA CCA GAG | Forward | 17 |
| | C-3′ | | |
| | 5′-CTG CAG CAG CCT CCT TCA | Reverse | 18 |
| | TGG C-3′ | | |
| Ifnb | 5′-GTC AGA GTG GAA ATC CTA | Forward | 19 |
| | AG-3′ | | |
| | 5′-ACA GCA TCT GCT GGT TGA | Reverse | 20 |
| | AG-3′ | | |
| Tnfα | 5′-CCA ACT GTC ACT CAT TGC | Forward | 21 |
| | TGA-3′ | | |
| | 5′-TTC CAA GAA GGA GAC CAT | Reverse | 22 |
| | GTT T-3′ | | |
| Il-6 | 5′-GCC CAG CTA TGA ACT CCT | Forward | 23 |
| | TCT-3′ | | |
| | 5′-GCG GCT ACA TCT TTG GAA | Reverse | 24 |
| | TCT-3′ | | |
| Ifit1 | 5′-GGA TTC TGT ACA ATA CAC | Forward | 25 |
| | TAG AAA CCA-3′ | | |
| | 5′-CTT TTG GTT ACT TTT CCC | Reverse | 26 |
| | CTA TCC-3′ | | |
| Ifit2 | 5′-ATC CCC CAT CGC TTA TCT | Forward | 27 |
| | CT-3′ | | |
| | 5′-CCACCTCAATTAATCAGGCACT- | Reverse | 28 |
| | 3′ | | |
| GAPDH | 5′-GCA AAT TCC ATG GCA CCG | Forward | 29 |
| | T-3′ | | |
| | 5′-TCG CCC CAC TTG ATT TTG | Reverse | 30 |
| | G-3′ | | |
| β- | 5′-AGA GCT ACG AGC TGC CTG | Forward | 31 |
| actin | AC-3′ | | |
| | 5′-CGT GGA TGC CAC AGG ACT- | reverse | 32 |
| | 3′ | | |

TABLE 5

| Step | Temperature | Time | | Cycle |
|---|---|---|---|---|
| Step 1 | 50.0° C. | 2 | mins | 1X |
| | 95.0° C. | 10 | mins | |
| Step 2 | 95.0° C. | 15 | secs | 40X |
| | 65.0° C. | 1 | min | |
| Step 3 | 95.0° C. | 15 | secs | 1X |
| | 65.0° C. | 1 | min | |
| | 95.0° C. | 1 | sec | |

### (5) Western Blot

Cells were lysed using a radioimmunoprecipitation assay (RIPA) buffer (Cell Signaling Technology® cat #9806) and a protease/phosphatase inhibitor mixture (Cell Signaling Technology®, cat #5872). The lysed cells were electrophoresed on 12% and 15% acrylamide gels using sodium dodecyl sulfate polyacrylamide gels at 80 to 120 V for 90 minutes.

The gels were transblotted using polyvinylidene difluoride (PVDF) membranes (Millipore Ltd., cat #617203) at 30 V for 90 minutes. Proteins were transferred to a PVDF membrane and blocked at room temperature for one hour using Tris-buffered saline (TBS) and 0.1% Tween-20 (TBS-T, Bio-Rad Laboratories, Inc., cat #1706531) and 5% skim milk. Next, the membrane was washed with TBS-T and cultured overnight in TBS-T at 4° C. with primary antibodies (SARS-CoV-2 nucleocapsid (Invitrogen™ cat #PA1-41098), caspase-3 (Cell Signaling Technology®, cat #9662), cleaved caspase-3 (Cell Signaling Technology®, cat #9664), mixed lineage kinase domain like pseudokinase (MLKL, Cell Signaling Technology®, cat #14993), phospho-MLKL (p-MLKL, Cell Signaling Technology®, cat #91689), mitofusin 1 (MFN1, Cell Signaling Technology®, cat #14739), mitofusin 2 (MFN2, Cell Signaling Technology®, cat #83667), translocase of the outer membrane 20 (TOM20, Cell Signaling Technology®, cat #72610), and glyceraldehyde 3-phosphate dehydrogenase (GAPDH, Sigma, cat #G9545)). Thereafter, the cells were washed three times with TBS-T and treated with a secondary antibody (Jackson ImmunoResearch Inc., cat #111-035-003) for one hour at room temperature. The antibody-treated PVDF membrane was detected with a horseradish peroxidase (HRP) substrate (EMD Millipore Corporation) using VILBER (FUSION Solo S instrument).

In the drawings showing the Western blot results, “+” means that the material was treated, and “−” means that the material was not treated.

### (6)RNA Sequencing Analysis and Differential Expression Gene (DEG) Analysis

To prepare a sequencing library of the extracted RNA, spliced reads were mapped to the human reference genome version (GRCh38) using the Bowtie2 [Reference 2] and HISAT2 programs [Reference 1] and then subjected to statistical processing. The number of processed and mapped reads for each sample was confirmed, and transcriptome assembly was performed using the reference gene model of the StringTie software program [Reference 3]. Afterward, the amount of transcripts was calculated by a read count, and calculated by fragments per kilobase of transcript per million mapped reads (FPKM) and transcripts per kilobase million (TPM) values.

A DEG analysis was performed on the raw data by targeting the read count values for known genes (obtained using the StringTie-e option) to filter out low-quality genes and using the edgeR R library-calcNormFactors to calculate the trimmed mean of M-value (TMM) normalization conditions (adjusted p-value<0.05; hypergeometric testing and multiple testing correction (false discovery rate, FDR) and fold change |Fold change (Fc)|≥2).

After transcriptome sequencing in two or more samples through the DEG analysis, differences in gene expression and regulation patterns between the sample groups were compared and modeled as a heat map.

In the drawings showing the results of the DEG analysis, “+” means that the material was treated, and “−” means that the material was not treated.

### (7) Mitochondrial Membrane Potential Measurement

Mitochondrial membrane potential probes MitoTracker™ Orange CMTMRos (Invitrogen™ cat #M7510) and JC-1 (Thermo Fisher Scientific, cat #T3168) were used to measure the recovery of mitochondrial membrane potential. Mitotracker™MOrange CMTMRos is a fluorescent dye that stains mitochondria in living cells and can be observed under a confocal microscope at a wavelength of 554 to 576 nm.

Calu-3 cells were seeded on a 4-chamber slide, infected with a virus and treated with Example Compound 1, and then treated with Mitotracker™MOrange CMTMRos at a final concentration of 500 nM in serum-free DMEM for 20 minutes at 37° C. and 5% CO₂. Control cells were treated with carbonyl cyanide-p-trifluoromethoxyphenylhydrazone (FCCP) for 20 minutes. All steps were carried out with light blocked, the 4-chamber slide was washed three times with pre-heated PBS, and an immunofluorescence analysis (IFA) was performed.

JC-1 is a marker for mitochondrial membrane potential, and appears as a green fluorescent monomer (˜529 nm) at depolarization and abnormal mitochondrial membrane potential. JC-1 was diluted to 10 mM, and the JC-1 solution was treated at a final concentration of 2 μM in serum-free DMEM for 20 minutes at 37° C. and 5% CO₂. Control cells were treated with H₂O₂ for 20 minutes. After JC-1 staining, nuclei were stained using Hoechst 33342 (Thermo Fisher Scientific, cat #62249) for five minutes at room temperature. After each staining step, cells were washed three times with pre-heated PBS and mounted on a glass slide. All steps were carried out with light blocked.

### Experimental Results

### Experimental Example 1. Confirmation of In Vitro Antiviral Efficacy of Example Compound 1 by Concentration Based on SARS-CoV-2 B.1 Infection Model

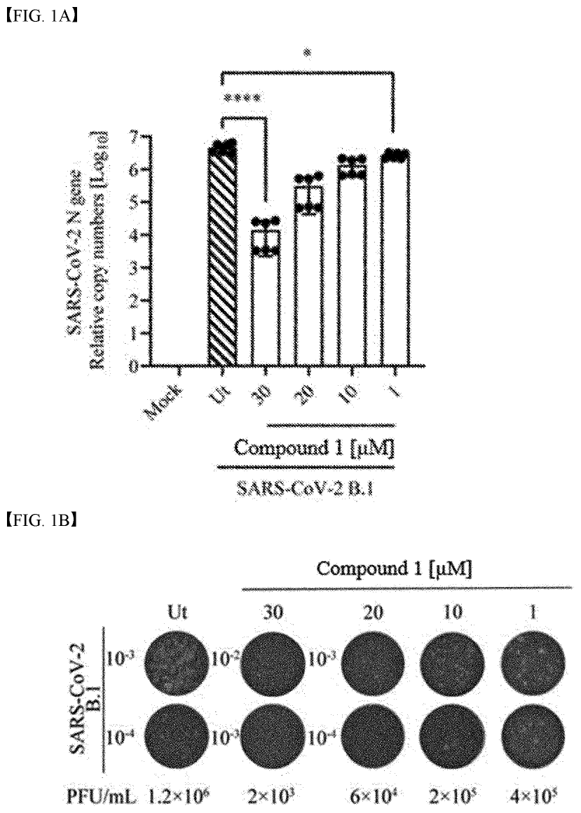

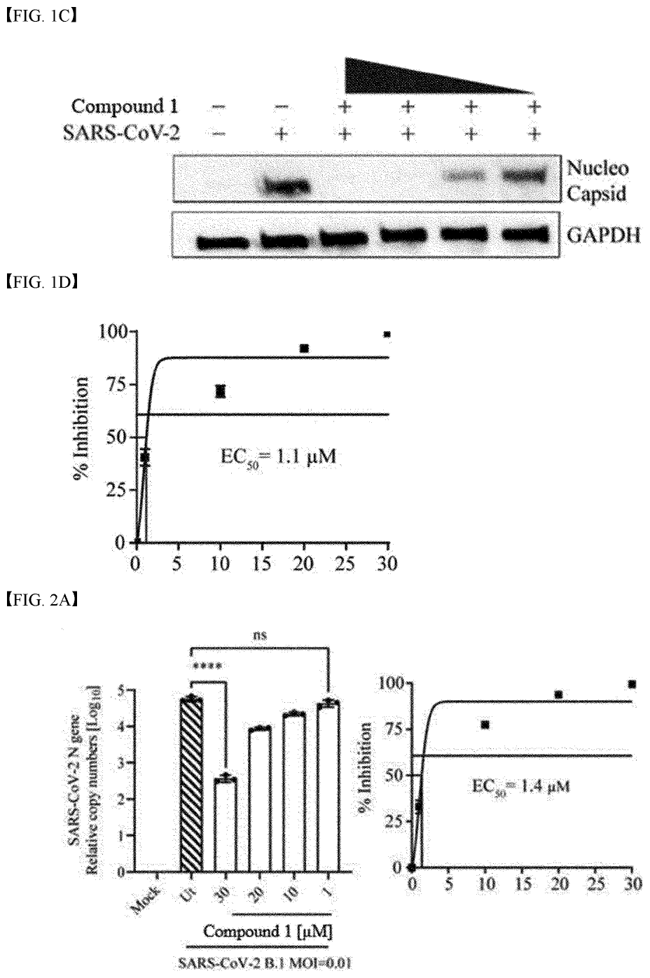

To investigate the antiviral activity of Example Compound 1 against SARS-CoV-2 B.1, Vero E6 cells were infected with SARS-CoV-2 B.1 (MOI=0.01). After 2 hours, Example Compound 1 was administered to the infected Vero E6 cells at each concentration of 30, 20, 10, and 1 μM. Total RNA, protein extracts, and supernatants were collected from the infected cells at 24 hours post-infection (hpi), and experiments were performed according to the above-described experimental method, and the results are shown in FIGS. 1A, 1B, 1C, and 1D.

FIGS. 1A, 1B, and 1C show the results of measuring antiviral activity through RT-qPCR, plaque assay, and Western blot, respectively. As shown in FIG. 1A, when the cells were treated with 30 μM of Example Compound 1, SARS-CoV-2 B.1 virus replication could be inhibited by up to 1,000 times compared to the case where the cells were not treated with Example Compound 1. As shown in FIG. 1B, when the cells were treated with 30 μM of Example Compound 1, the infectious particles of SARS-CoV-2 B.1 were reduced by 1,000 times compared to the case where the cells were not treated with Example Compound 1.

As shown in FIG. 1C, the expression of SARS-CoV-2 B.1 nucleocapsid protein also decreased in a dose-dependency of Example Compound 1, and it can be seen from FIG. 1D that the Example Compound 1 exhibited an EC₅₀ value of 1.1 μM against SARS-CoV-2 B.1.

It can be seen from the present Experimental Example 1 that Example Compound 1 exhibited antiviral efficacy in all RNA, protein, and viral molecule formation stages in Vero E6 cells infected with SARS-CoV-2 B.1, and that the replication of SARS-CoV-2 B.1 was gradually reduced in a dose-dependency of Example Compound 1.

### Experimental Example 2. Confirmation of In Vitro Antiviral Efficacy of Example Compound 1 Against SARS-CoV-2 B.1 Depending on the Concentration in hACE2-A549 Cells

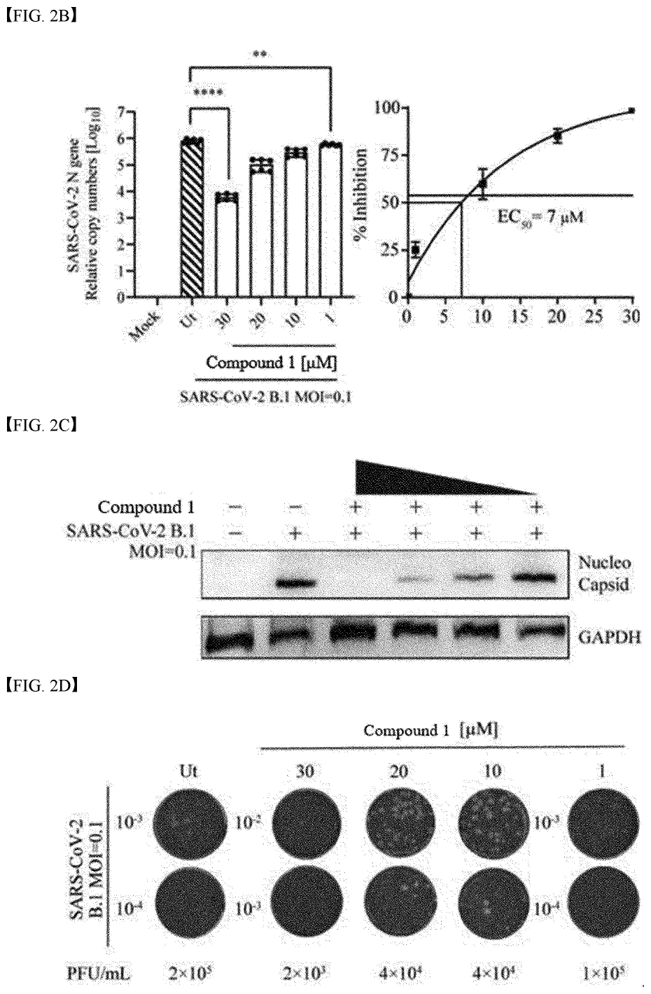

To investigate whether Example Compound 1 exhibits antiviral activity in human-derived cells, hACE2-A549 cells were infected with SARS-CoV-2 B.1 (MOI=0.01 and 0.1). After 2 hours, the infected hACE2-A549 cells were treated with Example Compound 1 at each concentration of 30, 20, 10, and 1 μM. Total RNA, protein extracts, and supernatants were collected from infected cells at 24 hours post-infection (hpi), and experiments were performed according to the above-described experimental method, and the results are shown in FIGS. 2A, 2B, 2C, and 2D

FIGS. 2A and 2B show the virus expression level at different concentrations of Example Compound 1 (left) and the EC₅₀ value of Example Compound 1 against SARS-CoV-2 B.1 virus (right) under MOI=0.01 and MOI=0.1 conditions.

As shown in FIG. 2A, under the MOI=0.01 condition, the antiviral activity of Example Compound 1 inhibited the replication of SARS-CoV-2 B.1 by about 100 times at a concentration of 30 UM and exhibited an EC₅₀ value of 1.4 μM against the SARS-CoV-2 B.1 virus. As shown in FIG. 2B, under the condition of MOI=0.1, the antiviral activity of Example Compound 1 inhibited the replication of SARS-CoV-2 B.1 by about 100 times at a concentration of 30 UM and exhibited an EC₅₀ value of 7 μM against the SARS-CoV-2 B.1 virus. In the case of infection under the MOI=0.01 and 0.1 conditions, the inhibitory rate at a concentration of 30 μM of Example Compound 1 was similar, but the EC₅₀ was about 5-fold different. The reason is that when the MOI=0.1 condition is used, the recovery speed of the virus is faster at low concentrations.

As shown in FIG. 2C, the expression of SARS-CoV-2 B.1 nucleocapsid protein decreased in a dose-dependency of Example Compound 1 under the MOI=0.1 condition, and as shown in FIG. 2D, infectious particles of SARS-CoV-2 B.1 were 100-fold reduced by the treatment with 30 μM of Example Compound 1 under the MOI=0.1 condition.

From the present Experimental Example 2, it can be seen that Example Compound 1 exhibited antiviral efficacy in all RNA, protein, and viral molecule formation stages in human-derived hACE2-A549 cells infected with SARS-CoV-2 B.1, and that the replication of SARS-CoV-2 B.1 was gradually reduced in a dose-dependency of Example Compound 1.

### Experimental Example 3. Analysis of Inflammatory Cytokines after Treatment with Example Compound 1 in hACE2-A549 Cells Infected with SARS-CoV-2 B.1

The expression of inflammatory cytokines was evaluated after treating SARS-CoV-2 B.1-infected hACE2-A549 cells with Example Compound 1.

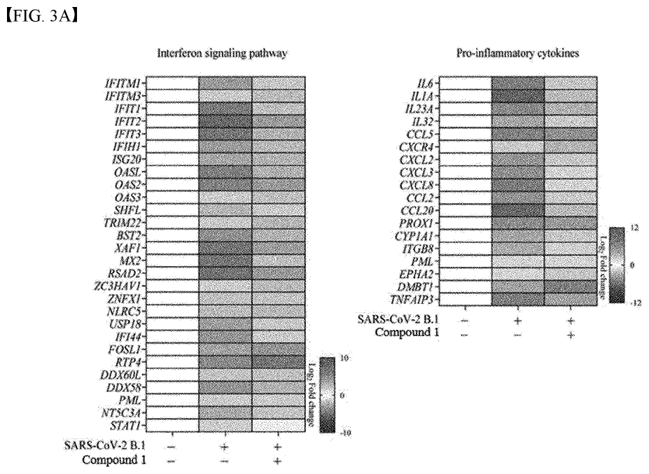

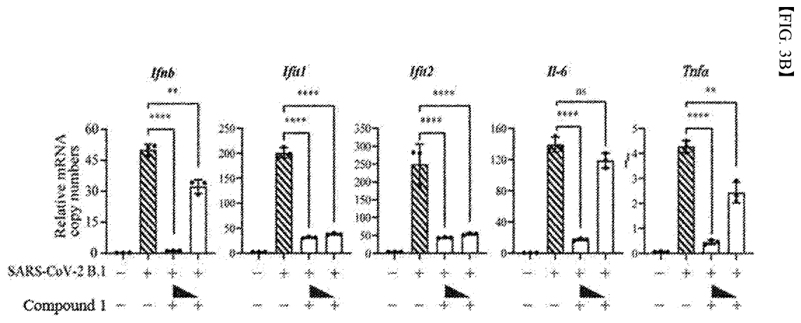

First, mRNA sequencing was performed through next-generation sequencing (NGS) and DEG analysis (the comparative combination satisfied the conditions |fc|≥2 & p-value<0.05), and the results are shown in FIG. 3A. From the results of the mRNAseq analysis of inflammatory cytokine-related genes shown in FIG. 3A, it can be seen that the interferon (IFN) signaling pathway and pro-inflammatory cytokines genes were highly induced by SARS-CoV-2 B.1 infection. In addition, it can be seen that treatment with Example Compound 1 inhibited the expression of the interferon signaling and pro-inflammatory cytokine genes.

From FIG. 3B, the expression level of the inflammatory cytokine-related genes can be confirmed by RT-qPCR, and it can be seen that the expression of interferon-related genes Ifnβ, Ifit1, and Ifit2 was significantly down-regulated by treatment with Example Compound 1 at 30 μM and 1 μM. In addition, treatment with Example Compound 1 at 30 μM and 1 μM reduced the induction of pro-inflammatory cytokine genes Il-6 and Tnfα. From this, it can be seen that the expression of pro-inflammatory cytokines was inhibited by the treatment of SARS-CoV-2 B.1-infected hACE2-A549 cells with Example Compound 1.

From Experimental Example 3, it can be seen that Example Compound 1 inhibits the expression of interferon and pro-inflammatory cytokines in SARS-CoV-2 B.1-infected hACE2-A549 cells.

### Experimental Example 4. Analysis of Nrf2 and Oxidative Phosphorylation (OXPHOS) Pathway after Treatment with Example Compound 1

The expression of nuclear factor erythroid-2-related factor 2 (Nrf2)-related genes and the OXPHOS pathway induced by treatment with Example Compound 1 was investigated.

First, hACE2-A549 cells were infected with SARS-CoV-2 B.1 at an MOI of 0.1. After two hours, the cells were treated with 30 μM of Example Compound 1, and total RNA was collected from the infected cells at 24 hours post-infection (hpi).

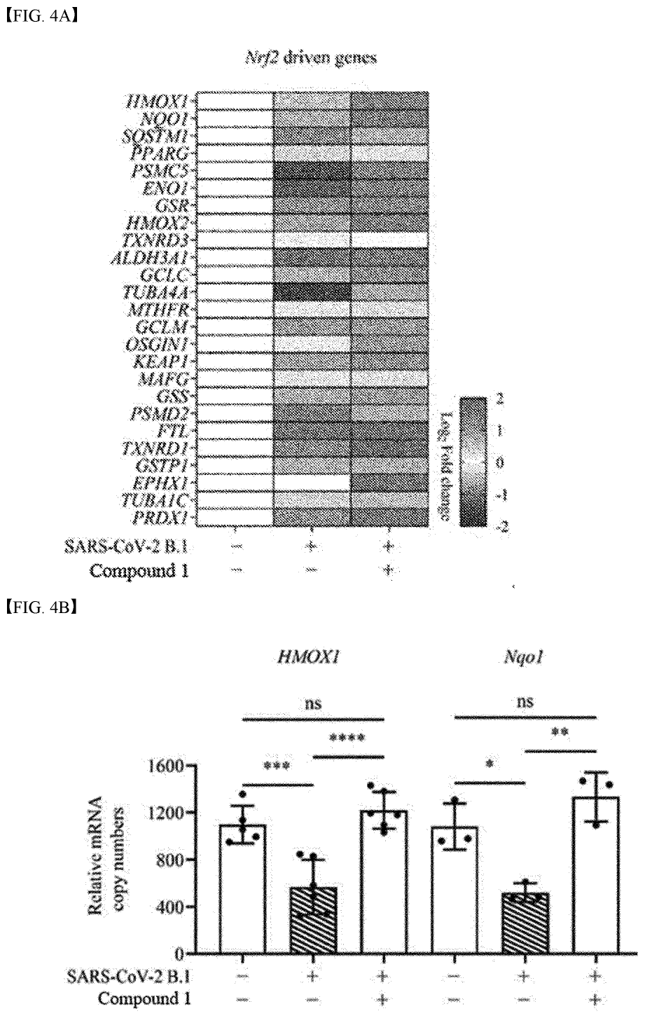

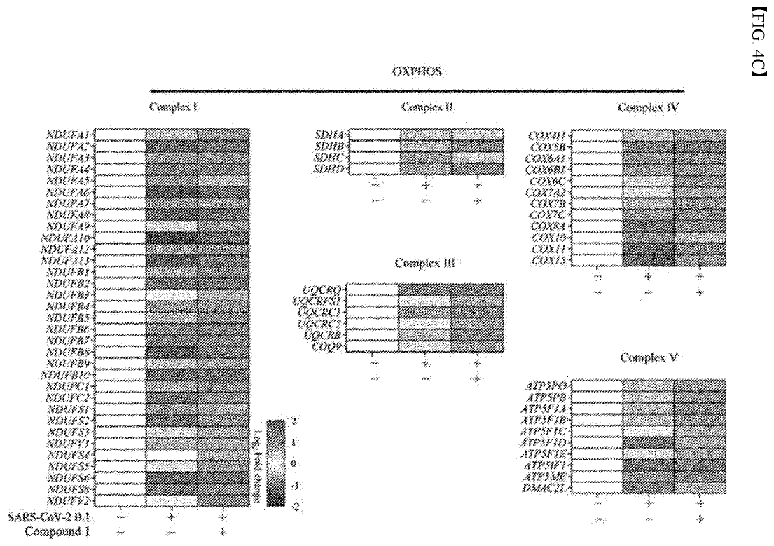

A DEG analysis was performed on the mRNAseq data, and the results are shown in FIG. 4A. As shown in FIG. 4A, it can be observed that the Nrf2 signaling pathway was down-regulated in the hACE2-A549 cells infected with SARS-CoV-2 B.1, and the Nrf2 signaling pathway was up-regulated by treatment with Example Compound 1.

The expression of the Nrf2-induced genes heme oxygenase 1 (HMOX1) and Nqo1 enables strong antioxidant activity and inhibition of the expression of inflammatory cytokines in cells. The results of analyzing the gene expression of HMOX1 and Nqo1 using RT-qPCR after treatment with Example Compound 1 are shown in FIG. 4B, and compared to the infected cells that were not treated with Example Compound 1, the infected cells treated with Example Compound 1 exhibited a significant increase in the expression of the above two genes, and their expression levels were similar to those in cells that were not infected with a virus.

Most intracellular ATP is produced by the mitochondrial OXPHOS process. After treatment with Example Compound 1, changes in the expression level of the mitochondrial OXPHOS complex were evaluated through a DEG analysis of mRNAseq data, and the results are shown in FIG. 4C. SARS-CoV-2 B.1 infection decreased the expression levels of mitochondrial OXPHOS complexes I, II, III, IV, and V, but the cells treated with Example Compound 1 exhibited up-regulated expression of all five OXPHOS complexes, compared to the cells infected with SARS-CoV-2. B.1. The treatment of SARS-CoV-2 B.1-infected hACE2-A549 cells with Example Compound 1 up-regulated the expression of HMOX1 and Nqo1 in the Nrf2 signaling pathway and OXPHOS-related genes, suggesting that Example Compound 1 promotes the recovery of mitochondrial function after SARS-CoV-2 B infection.

From the present Experimental Example 4, it can be seen that Example Compound 1 up-regulated the expression of the intracellular antioxidant transcription factor Nrf2 and the expression of mitochondrial OXPHOS, which were inhibited by SARS-CoV-2 B.1 infection in hACE2-A549 cells.

### Experimental Example 5. Evaluation of Mitochondrial Homeostasis Maintenance in hACE2-A549 Cells and Calu-3 Cells Infected with SARS-CoV-2 B.1 after Treatment with Example Compound 1

To evaluate mitochondrial homeostasis by treatment with Example Compound 1, hACE2-A549 cells and Calu-3 cells were infected with SARS-CoV-2 B.1 virus at MOI=0.1 and 1. After two hours, the cells were treated with Example Compound 1 at 30 μM, and JC-1 and MitoTracker staining of the hACE2A-549 cells and Calu-3 cells was performed at 24 hpi.

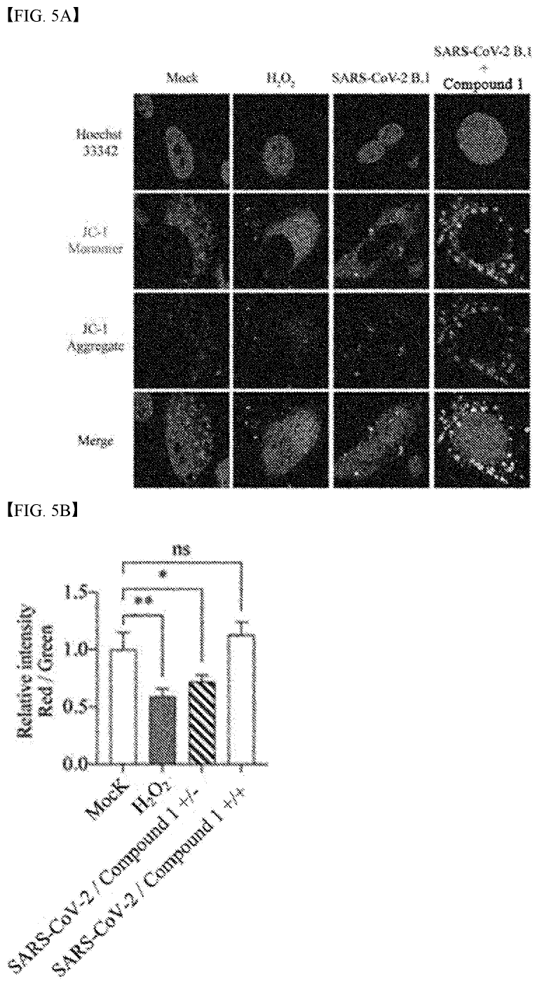

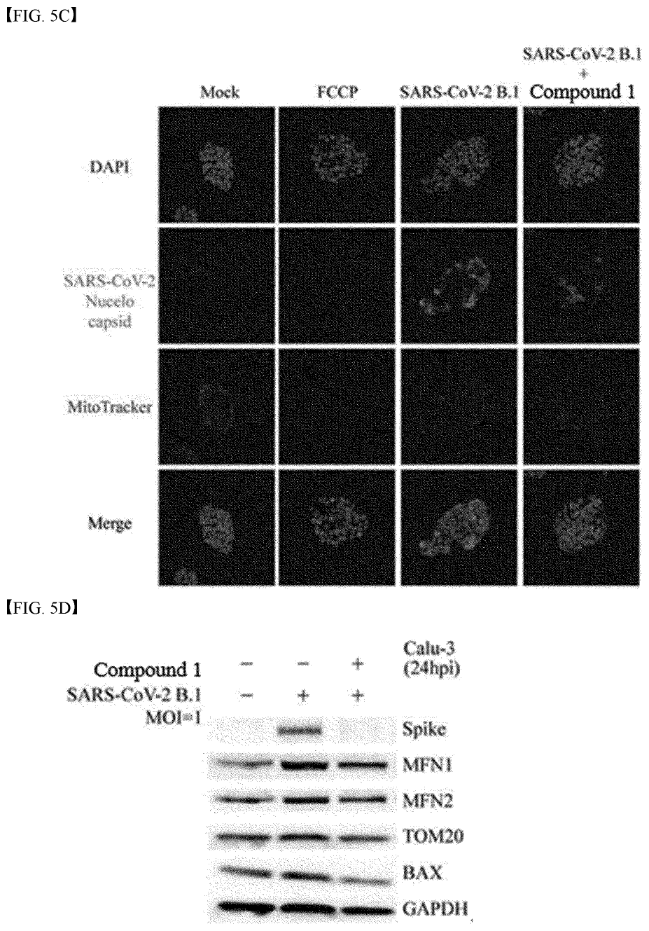

JC-1 staining was performed to detect dynamic changes in mitochondrial membrane potential (AY) when hACE2-A549 cells were infected with the SARS-CoV-2 B.1 strain, and the impact of Example Compound 1 on membrane potential recovery was observed at 24 hpi, and the results are shown in FIGS. 5A and 5B.

The mitochondrial membrane potential (JC-1 Aggregate, RED) of hACE2-A549 cells decreased after SARS-CoV-2 B.1 infection, and treatment of the infected cells with Example Compound 1 resulted in an intensity level similar to that of the uninfected cells at 24 hpi.

In addition, after infecting the Calu-3 cells with SARS-CoV-2, the membrane potential of the infected cells was measured using MitoTracker™ Orange CMTMRos and IFA, and the results are shown in FIG. 5C. The accumulation of MitoTracker was significantly reduced in SARS-CoV-2 B.1-infected cells at 24 hpi. Treatment with Example Compound 1 resulted in the accumulation of MitoTracker and a significant decrease in SARS-CoV-2 nucleocapsid protein.

From the JC-1 and MitoTracker staining results, it can be observed that the membrane potential of intracellular mitochondria, maintained by proton pumps (complexes I, III, and IV) during the energy storage process in the oxidative phosphorylation process, was decreased in response to SARS-CoV-2 infection and recovered after treatment with Example Compound 1.

Proteins from the Calu-3 cells infected with the SARS-CoV-2 B.1 strain were collected at 24 hpi using RIPA buffer containing phosphotease and protease inhibitors. Mitochondrial dynamics, especially the fusion-related factors MFN1 and MFN2, were observed at the protein level, and the results are shown in FIG. 5D. In the Calu-3 cells infected with SARS-CoV-2 B.1, both MFN1 and MFN2 were overexpressed, and the overexpression was suppressed after treatment with Example Compound 1.

The results of the present Experimental Example 5 suggest that mitochondria are under continuous stress due to SARS-CoV-2 infection and that mitochondrial stress is suppressed by treatment with Example Compound 1.

### Experimental Example 6. Cell Death Pattern Analysis of hACE2-A549 Infected with SARS-CoV-2 B.1 after Treatment with Example Compound 1

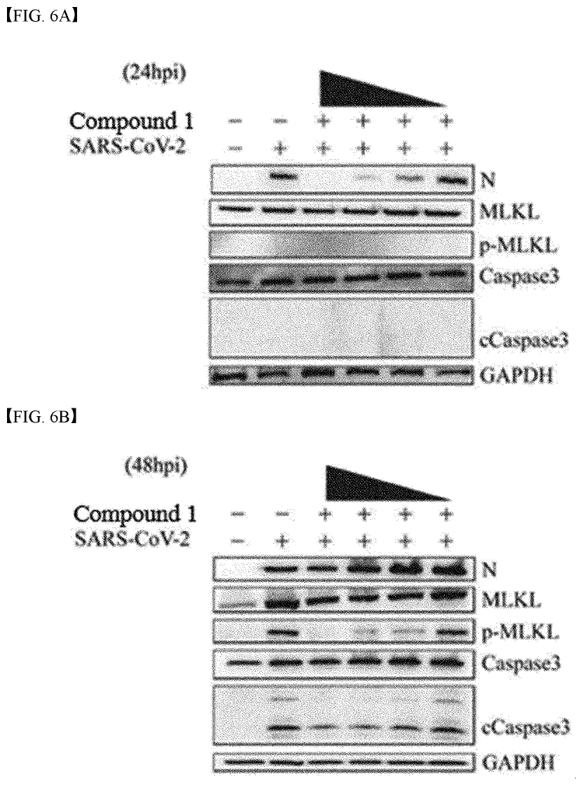

To evaluate the pattern of mitochondrial cell death by treatment with Example Compound 1, hACE2-A549 cells were infected with SARS-CoV-2 B.1 at an MOI of 0.1. After two hours, the cells were treated with Example Compound 1 at each of 30, 20, 10, and 1 μM. Western blot was used to evaluate the pattern of cell death caused by mitochondrial dysfunction, and the results are shown in FIGS. 6A and 6B.

As shown in FIG. 6A, cell death-related factors cleaved-caspase-3 (cCaspase3) and phospho-MLKL (p-MLKL) could not be detected at 24 hpi. However, as shown in FIG. 6B, at a concentration of 30 μM, necrosis-related p-MLKL protein and cell death-related cCaspase 3 protein were inhibited at 48 hpi.

Accordingly, it can be seen from the present Experimental Example 6 that Example Compound 1 inhibits the expression of cell death-related proteins caused by SARS-CoV-2 B.1.

### Experimental Example 7. Confirmation of Universal Antiviral Efficacy of Example Compound 1

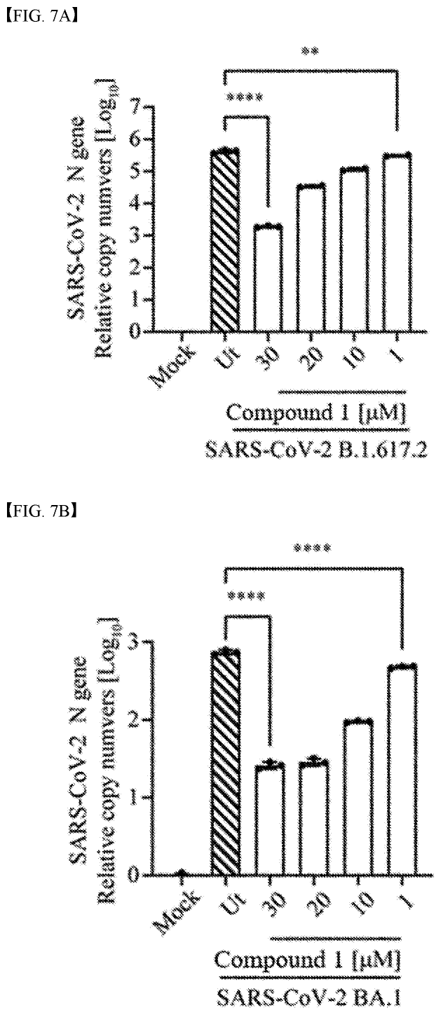

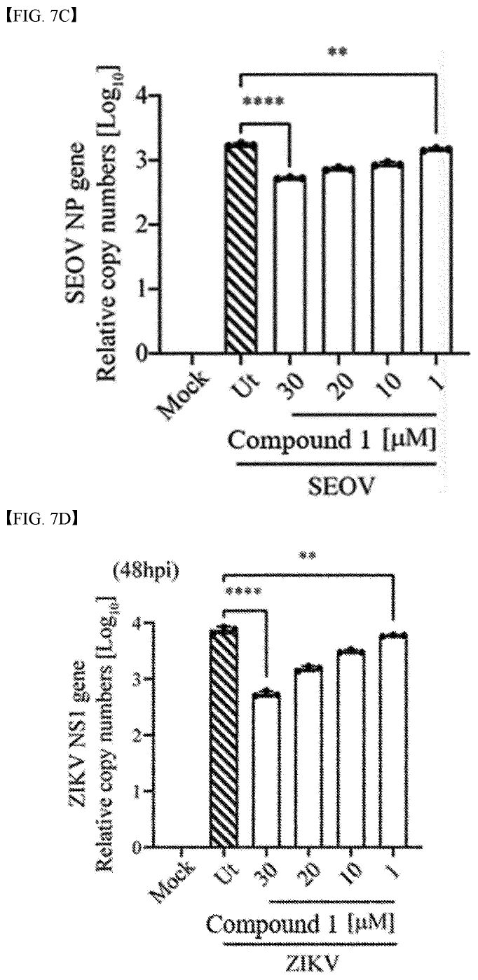

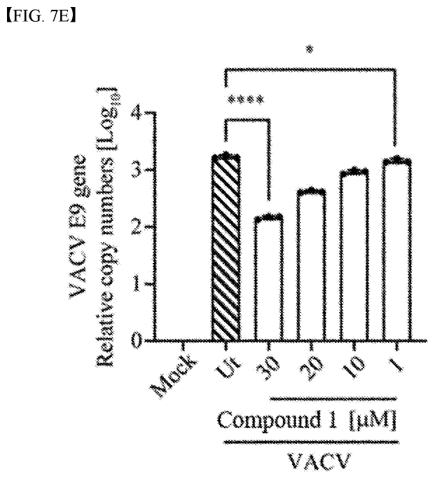

The antiviral activity of Example Compound 1 against SARS-CoV-2 variants and other viruses was evaluated to examine whether its antiviral activity is limited to SARS-CoV-2 B.1 and may be extended to other human pathogenic viruses. The hACE2-A549 cells and Vero E6 cells were infected with SARS-CoV-2 B.1.617.2 (delta), BA.1 (omicron), Seoul virus (SEOV), Zika virus (ZIKV), and vaccinia virus (VACV), respectively. After two hours, Example Compound 1 was administered to the infected cells at each concentration of 30, 20, 10, and 1 μM. Total RNA was collected from the infected cells at 24 hpi, except for ZIKV. RT-qPCR was performed on the collected RNA, and the results are shown in FIGS. 7A, 7B, 7C, 7D, and 7E for each virus.

As shown in FIGS. 7A and 7B, Example Compound 1 exhibited about 1000-fold and 30-fold antiviral effects in the hACE2-A549 cells infected with SARS-CoV-2 B.1.617.2 (delta) and BA.1 (omicron), respectively. As shown in FIG. 7C, treatment with Example Compound 1 reduced the expression level of SEOV by about 3 times in the Vero E6 cells. As shown in FIGS. 7D and 7E, treatment with Example Compound 1 in the Vero E6 cells reduced the expression level of ZIKV by about 16 times and reduced the expression level of VACV by about 15 times.

In addition, all viruses were reduced in a dose-dependent manner, and the EC₅₀ value of Example Compound 1 for each virus is shown in Table 6. These results suggest that Example Compound 1 is a broad-spectrum antiviral agent against SARS-CoV-2 variants and various viruses.

TABLE 6

| Type | Classification | Virus | EC₅₀ [μM] |
|---|---|---|---|
| RNA | Coronaviridae | SARS-CoV-2 | 1.2 |
| | | B.1.617.2 | |
| | | SARS-CoV-2 | 1.1 |
| | | BA.1 | |
| | Bunyaviridae | SEOV | 1.9 |
| | Flaviviridae | ZIKV | 7.8 (48 hpi) |
| DNA | Poxviridae | VACV | 6.6 |

### REFERENCES

[1] Kim, Daehwan, et al. “Graph-based genome alignment and genotyping with HISAT2 and HISAT-genotype.” Nature Biotechnology 37.8 (2019): 907-915.
[2] Langmead, Ben, and Steven L. Salzberg. “Fast gapped-read alignment with Bowtie 2.” Nature Methods 9.4 (2012): 357-359.
[3] Pertea, Mihaela, et al. “StringTie enables improved reconstruction of a transcriptome from RNA-seq reads.” Nature Biotechnology 33.3 (2015): 290-295.

1. A method of preventing or treating viral infections, comprising:
administering a compound of Chemical Formula 1 or a pharmaceutically acceptable salt thereof to a subject in need thereof: 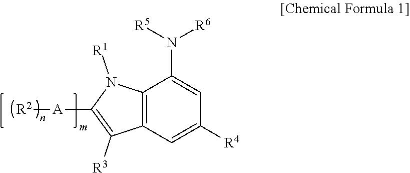
wherein:
n is an integer from 1 to 3,
m is 0 or 1,
A is phenyl,
R¹ is hydrogen or C₁-C₆ alkyl,
R² is hydrogen, halogen, C₁-C₆ alkoxy, —C₁-C₆ alkylene-OH, —(CH₂)ₚCO₂R⁷, —NHR⁸, —N(H)S(O)₂R⁷, or —NHC(O)R⁷, wherein p is an integer from 0 to 3, R⁷ is hydrogen or C₁-C₃ alkyl, and R⁸ is C₁-C₃ alkylpiperidinyl or C₁-C₃ alkylsulfonyl,
R³ is hydrogen, halogen, C₁-C₆ alkyl, phenyl, or —(CH₂)ₚ-heterocycle, in which the heterocycle is a 5- or 6-membered ring including 1 or 2 hetero atoms selected from S, N, and O atoms, wherein p is an integer from 0 to 3, provided that when m is 0, R³ is phenyl,
R⁴ is halogen, C₁-C₆ alkyl, —C₁-C₆ alkylene-OH, —O-phenyl, —(CH₂)ₚCO₂R⁷, —(CH₂)ₚ-heterocycle, in which the heterocycle is a 5- or 6-membered ring including 1 or 2 hetero atoms selected from S, N, and O atoms, or proline-N-carbonyl wherein p is an integer from 0 to 3, and R⁷ is the same as defined above,
R⁵ is hydrogen or C₁-C₆ alkyl,
R⁶ is C₁-C₆ alkyl, C₃-C₆ cycloalkyl, heterocycle, or —C₁-C₆ alkylene-heterocycle, in which the heterocycle is a 3- to 8-membered ring including 1 to 3 hetero atoms selected from S, N, and O atoms, and R⁶ may be substituted with C₁-C₆ alkylamine, —C₁-C₆ alkylene-OH, or C₁-C₆ alkylsulfonyl.

2. The method of claim 1, wherein R³ is hydrogen, halogen, phenyl, or —(CH₂)ₚ-heterocycle in which the heterocycle is morpholino or piperazinonyl, and p is an integer of 0 or 1, provided that when m is 0, R³ is phenyl,
R⁴ is halogen, C₁-C₃ alkyl, —C₁-C₃ alkylene-OH, —O-phenyl, —(CH₂)ₚCO₂-ethyl, —(CH₂)ₚ-heterocycle in which the heterocycle is thiomorpholino, morpholino, piperazinonyl, or pyrrolidinyl, or proline-N-carbonyl, wherein p is an integer of 0 or 1,
R⁵ is hydrogen or C₁-C₃ alkyl,
R⁶ is C₁-C₃ alkyl, C₃-C₆ cycloalkyl, heterocycle, or —C₁-C₃ alkylene-heterocycle, wherein the heterocycle is tetrahydro-2H-pyran, or piperidinyl, and when R⁶ is heterocycle or —C₁-C₃ alkylene-heterocycle, the heterocycle may be substituted with C₁-C₆ alkylamine, —C₁-C₆ alkylene-OH, or C₁-C₆ alkylsulfonyl.

3. The method of claim 1, wherein the compound of Chemical Formula 1 is any one selected from the group consisting of the following compounds:
<1> 5-[(1,1-dioxido-4-thiomorpholinyl)methyl]-2-phenyl-N-(tetrahydro-2H-pyran-4-yl)-1H-indol-7-amine; <2> ethyl 7-(cyclopentylamino)-2-phenyl-1H-indol-5-carboxylate; <3> (7-(cyclopentylamino)-2-phenyl-1H-indol-5-yl) methanol; <4> 5-chloro-N,1-dimethyl-2-phenyl-N-(tetrahydro-2H-pyran-4-yl)-1H-indol-7-amine; <5> 4-((7-(cyclopentylamino)-2-(3-fluorophenyl)-1H-indol-5-yl)methyl)piperazin-2-one; <6> 4-((2-phenyl-7-(((tetrahydro-2H-pyran-4-yl)methyl)amino)-1H-indol-5-yl)methyl)thiomorpholine 1,1-dioxide; <7> 5-chloro-N-(1-methylpiperidin-4-yl)-2-phenyl-1H-indol-7-amine; <8> 5-phenoxy-2-phenyl-N-(tetrahydro-2H-pyran-4-yl)-1H-indol-7-amine; <9> 5-chloro-3-(morpholinomethyl)-2-phenyl-N-(tetrahydro-2H-pyran-4-yl)-1H-indol-7-amine; <10> 2-(4-((5-fluoro-2-phenyl-1H-indol-7-yl)amino)piperidin-1-yl) ethan-1-ol; <11> N-(4-(5-chloro-7-(cyclopentylamino)-1H-indol-2-yl)phenyl)methanesulfonamide; <12> 5-chloro-3-phenyl-N-(tetrahydro-2H-pyran-4-yl)-1H-indol-7-amine; <13> 4-((2-(3-fluorophenyl)-7-((tetrahydro-2H-pyran-4-yl)amino)-1H-indol-5-yl)methyl)thiomorpholine 1,1-dioxide; <14> 5-chloro-N-cyclopentyl-2-(4-((1-methylpiperidin-4-yl)amino)phenyl)-1H-indol-7-amine; <15> 4-((7-(isopentylamino)-2-(4-methoxyphenyl)-1H-indol-5-yl)methyl)thiomorpholine 1,1-dioxide; <16> N-(4-(7-(cyclopentylamino)-5-((1,1-dioxidothiomorpholino)methyl)-1H-indol-2-yl)phenyl)acetamide; <17> 4-((3-bromo-2-phenyl-7-((tetrahydro-2H-pyran-4-yl)amino)-1H-indol-5-yl)methyl)thiomorpholine 1,1-dioxide; <18> 4-((5-chloro-2-phenyl-7-((tetrahydro-2H-pyran-4-yl)amino)-1H-indol-3-yl)methyl)piperazin-2-one; <19> 4-((7-(methyl(tetrahydro-2H-pyran-4-yl)amino)-2-phenyl-1H-indol-5-yl)methyl)thiomorpholine 1,1-dioxide; <20> 5-methyl-N-(1-(methylsulfonyl)piperidin-4-yl)-2-phenyl-1H-indol-7-amine; <21> N-(4-(5-(1,1-dioxidothiomorpholino)-7-((tetrahydro-2H-pyran-4-yl)amino)-1H-indol-2-yl)phenyl)acetamide; <22> 4-((7-((1-(methylsulfonyl)piperidin-4-yl)amino)-2-phenyl-1H-indol-5-yl)methyl)thiomorpholine 1,1-dioxide; <23> N¹-(5-chloro-2-phenyl-1H-indol-7-yl)-N⁴-methylcyclohexane-1,4-diamine; <24> methyl 2-(3-(5-chloro-7-((tetrahydro-2H-pyran-4-yl)amino)-1H-indol-2-yl)phenyl)acetate; <25> (2-phenyl-7-((tetrahydro-2H-pyran-4-yl)amino)-1H-indol-5-carbonyl)-D-proline; <26> (3-(5-chloro-7-((tetrahydro-2H-pyran-4-yl)amino)-1H-indol-2-yl)phenyl)methanol; <27> N-cyclopentyl-2-phenyl-5-(2-(pyrrolidin-1-yl)ethyl)-1H-indol-7-amine; <28> methyl 2-(4-(5-chloro-7-((tetrahydro-2H-pyran-4-yl)amino)-1H-indol-2-yl)phenyl)acetate; <29> methyl 4-(5-chloro-7-(cyclopentylamino)-1H-indol-2-yl)benzoate; <30> 2-(4-(5-chloro-7-(tetrahydro-2H-pyran-4-yl)amino)-1H-indol-2-yl)phenyl) ethan-1-ol; <31> 3-bromo-5-(morpholinomethyl)-2-phenyl-N-(tetrahydro-2H-pyran-4-yl)-1H-indol-7-amine; and <32> 4-((3-phenyl-7-((tetrahydro-2H-pyran-4-yl)amino)-1H-indol-5-yl)methyl)thiomorpholine 1,1-dioxide.

4. The method of claim 1, wherein the compound of Chemical Formula 1 is a compound of Chemical Formula 2 below.

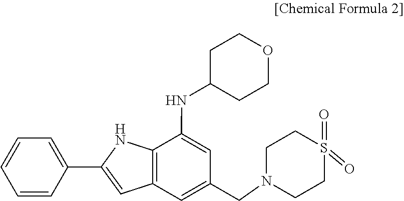 [SMILES: O=S1(=O)CCN(Cc2cc(NC3CCOCC3)c3[nH]c(-c4ccccc4)cc3c2)CC1]

5. The method of claim 1, wherein the method is for prevention or treatment of one or more viral infections selected from the group consisting of severe acute respiratory syndrome coronavirus type 2 (SARS-CoV-2), severe acute respiratory syndrome coronavirus type 1 (SARS-CoV-1), Zika virus (ZIKV), Gamak virus (GAKV), respiratory syncytial virus (RSV), vaccinia virus (VACV), influenza virus, flavivirus, adenovirus (AdV), Middle East respiratory syndrome coronavirus (MERS-CoV), herpes virus, Japanese encephalitis virus (JEV), Epstein-Barr virus (EBV), Ebola virus (EBOV), rhinovirus, chikungunya virus (CHIKV), hepatitis A virus (HAV), hepatitis B virus (HBV), hepatitis C virus (HCV), rotavirus, astrovirus, hantavirus including Seoul virus (SEOV), dengue virus, severe fever with thrombocytopenia syndrome virus (SFTSV), human immunodeficiency virus (HIV), West Nile virus (WNV), and yellow fever virus, and variants thereof.

6. The method of claim 1, wherein the method is for prevention or treatment of coronavirus disease 2019 (COVID-19).

7. The method of claim 1, wherein the method increases the expression of one or more genes selected from the group consisting of HMOX1 and Nqo1, which is inhibited due to viral infection or inhibits the expression of one or more genes selected from the group consisting of Ifnb, Tnfα, Il-6, Ifit1, and Ifit2, which is increased due to viral infection.

8. (canceled)
